(19) United States
(12) Patent Application Publication
(10) Pub. No.: US 2005/0031708 A1
(43) Pub. Date: Feb. 10, 2005

(54) Composition comprising a zeolite compound for treatment of diseases

(76) Inventor: Micah S. Portney, West Nyack, NY (US)

Correspondence Address: KLAUBER & JACKSON, 411 HACKENSACK AVENUE, HACKENSACK, NJ, 07601, US

(21) Appl. No.: 10/910,530

(22) Filed: Aug. 3, 2004

Related U.S. Application Data
(60) Provisional application No. 60/492,666, filed on Aug. 6, 2003.
(60) Provisional application No. 60/536,272, filed on Jan. 14, 2004.

(51) Int. Cl. A61K 33/04; A61K 33/06

(52) U.S. Cl. 424/684; 514/52; 424/702; 514/725; 514/167; 514/251; 514/276; 514/350; 514/355; 514/458; 514/475; 514/560

(57) ABSTRACT

The present invention relates to a composition comprising a zeolite useful for treating multiple conditions. The zeolite is normally present in a weight percent of about 50-95%. Optionally, one or more other ingredients are present in the composition. Such supplemental ingredients may include one or more of calcium, vitamin B-12, vitamin A, vitamin B1, vitamin B2, vitamin B3, vitamin B6, vitamin B7, folate, pantothenic acid, vitamin C, vitamin D, vitamin E, vitamin K, iron, zinc, magnesium, potassium, boron, iodine, manganese, selenium, copper, chromium, omega 3 or omega 6 fatty acids. Calcium is normally present in a weight percent of about 3-50%. Vitamin B-12 may be present in a weight percent of about 1-5%. The zeolite is normally a clinoptilolite zeolite or a heulandite zeolite, and it is usually present in the composition as a powder having an average particle size of about 1 to 2000 microns, often about 500 microns. The compositions described herein are useful for preventing or treating a disease such as the hangover effect of alcohol, diarrhea, heartburn, gastrointestinal disease, toxic poisoning, influenza, and the common cold. The compositions are further useful for detoxifying an organism or a biological sample.

### FIELD OF THE INVENTION

The present invention relates to a composition containing a zeolite useful for treating multiple conditions such as the hangover effect of alcohol, diarrhea, heartburn, gastrointestinal disease, toxic poisoning, influenza, and the common cold. The compositions are further useful for detoxifying an organism or a biological sample.

### BACKGROUND OF THE INVENTION

The zeolites are framework silicates consisting of interlocking tetrahedrons of SiO₄ and AlO₄. Zeolites have a (Si+Al)/O ration of ½. The alumino-silicate structure is negatively charged and attracts positive cations within. Zeolites have large vacant spaces or cages in their structures that allow space for large cations such as sodium, potassium, barium and calcium and even large molecules and cation groups such as water, ammonia, carbonate ions and nitrate ions. In some zeolites, the spaces are interconnected and form long wide channels of varying sizes depending on the mineral. These channels allow easy movement of resident ions and molecules into and out of the structure. Zeolites can lose and absorb water without damage to their crystal structures. The large channels explain the consistent low specific gravity of these minerals.

Zeolites have many useful purposes. They may be used for ion exchange, filtering, odor removal, as a chemical sieve and for gas absorption. They are frequently used in water softeners. Calcium in water may cause it to be “hard” and creates problems such as scum formation. Zeolites charged with sodium ions allow hard water to pass through and exchange the calcium for sodium ions in a reversable process. In a similar way zeolites absorb ions and molecules and thus act as a filter for odor control, toxin removal and as a chemical sieve. Zeolites may be dried or dehydrated by heat with the basic structure left intact. Following, other solutions may be delivered so that the zeolites can then act as a delivery system for the new fluid. This process has applications in medicine, livestock feeds and other types of research. Zeolites added to livestock feed have been shown to absorb toxins that are damaging and even fatal to the growth of the animals, while the basic structure of the zeolite is biologically neutral. Most municipal water supplies are processed through zeolites before public consumption

Clinoptilolite is a well known natural zeolite used in many of the foregoing applications. Commercially available natural zeolites are usually of the clinoptilolite (clino) variety having the general simplified chemical formula of (Na,K)₆Si₃₀Al₆)₇₂*nH₂0. It is well suited for these applications due to its large amount of pore space, high resistance to extreme temperatures and chemically neutral basic structure. It has been used for several years as a feed additive for cows, pigs, horses and chickens.

Clinoptilolite forms as a devitrification product (the conversion of glass to crystalline material) of volcanic glass in tuffs, or consolidated pyroclastic rocks. Such devitrification occurs when the glass is in contact with saline waters. Clinoptilolite is also found in the vesicles of volcanic rocks such as basalts, rhyolites and andesites. It forms as an alteration of phillipsite in deep-sea sediments and with borate minerals in playa lakes.

The structure of clinoptilolite is the same as heulandite (having a simplified chemical formula of Ca₄Al₈Si₂₈O₇₂*nH₂O) and has a sheet-like organizational structure. It differs from heulandite primarily in being enriched in potassium. The sheets are connected to each other by a few bonds that are relatively widely separated and contain open rings of alternating eight and ten sides. These rings stack together from sheet to sheet to form channels throughout the crystal structure. The size of these channels controls the size of the molecules or ions that can pass through them and therefore provides the capacity to function as a chemical sieve, allowing some ions to pass through while blocking others.

Some synthetic zeolites are now available. The simplest synthetic zeolite is the zeolite A with a molecular ratio of one silica to one alumina to one sodium cation. The zeolite A synthesis produces precisely duplicated sodalite units which have about 47% open space.

Certain medical applications for zeolite have been contemplated far removed from the industrial applications for which they have historically been used. For instance, Andersson et al., U.S. Pat. No. 5,936,061 describe using hydrophobic zeolites for removing preservatives from polypeptide solutions, for example those used for pharmaceutical preparations. Varga et al., U.S. Pat. No. 5,264,225 describe using zeolite in combination with other materials for removing radioactive agents in biological applications. Tanimoto et al., U.S. Pat. No. 6,071,542 describe an antibacterial zeolite composition. Bgatov et al., U.S. Pat. No. 6,287,576 describe a composition of biologically active vegetable stock and about 92% by weight clinoptilolite and/or montmorillonite clay. Rodriguez-Fuentes et al. describe a zeolite composition alone at about 55-70% weight percent for treating diarrhea. (Rodriguez-Fuentes et al, Zeolites 19:441-448 (1997). Moreover, Pavelic et al. investigate the potential immune stimulating effects of zeolites such as may be applied for use as an adjuvant. Pavelic et al., J. Mol. Med. (2001) 78:708-720.

Alcohol intoxication causes dehydration and an imbalance in electrolytes, minerals, and some vitamins in the bloodstream, disrupting many normal biological processes. Additionally, when the blood alcohol level is high, liver enzymes responsible for detoxification cannot function quickly enough so that toxic metabolites are produced. Such metabolites may be more toxic than alcohol and can cause nausea, headaches, and discomfort, usually collectively referred to as a “hangover” or more technically veisalgia. Congeners, toxic byproducts of distillation and fermentation, worsen hangovers. Some alcoholic beverages such as red wine, brandies, and whiskies have higher concentrations of congeners than others. There is a need for new remedies to relieve the deleterious side effects of excessive alcohol consumption.

Diarrhea is one of the most common health problems in the world, and even in developed countries is one of the most common infectious diseases. The medical community continues to seek improved methods and products for treating and preventing diarrhea. In recent years, rotavirus and other enteric viruses have been identified as a major cause of acute diarrhea in infants and young children. Additionally, Clostridium difficile is often involved in the pathogenesis of diarrhea. Other common causes of diarrhea include food intoxication, accumulation of bile acids and neurological impairment especially as associated with diabetes. There is a need for additional products and methods effective to prevent and treat diarrhea.

There are three known influenza-type viruses that affect human beings, namely, Influenza A, B and C. Influenza A viruses have been isolated from many animal species in addition to humans, while the influenza B and C viruses infect mainly humans. The influenza viruses are enveloped viruses containing negative single-stranded RNA that is segmented and encapsidated. The influenza virus envelope is characterized by the presence of two surface glycoproteins: hemagglutinin and neuraminidase. The influenza A and B virions are pleomorphic and are usually 80-120 nm in diameter. The influenza C virion has many distinctive properties and is thus distinguished from the closely related A and B virions. Infection with influenza A or B generally causes a highly contagious, acute respiratory illness.

Influenza viruses have a major impact on morbidity leading to hospitalization and health care provider visits. High rates of hospitalization are observed for patients over 65 years of age and children less than 5 years of age. Influenza virus is also unique among respiratory viruses in causing significant mortality. Furthermore, the spread of influenza virus through a population can result in epidemics of considerable economic impact. For example, high rates of mortality have been observed due to influenza infection during influenza epidemics such as those of 1957, 1968 and 1977. Fields Virology, Second Edition, Volume 1, pp. 1075-1152 (1990).

There are few known compounds that have significant anti-viral activity against influenza viruses. Two of these, amantadine and rimantadine are approved in the United States for treating influenza virus disease. Both compounds are most effective when used prophylactically as influenza viruses rapidly develop resistance to both compounds.

### SUMMARY OF THE INVENTION

In its broadest aspect, the invention relates to a composition of therapeutic utility comprising a zeolite. Preferably, the zeolite is present in a weight percent of about 50-95%, more preferably about 70-95%, more preferably still, about 90-95%. Optionally, one or more other ingredients are present in the composition. Such supplemental ingredients may include one or more of calcium, vitamin B-12, vitamin A, vitamin B1 vitamin B2, vitamin B3, vitamin B6, vitamin B7, folate, pantothenic acid, vitamin C, vitamin D, vitamin E, vitamin K, iron, zinc, magnesium, potassium, boron, iodine, manganese, selenium, copper, chromium, and omega 3 or omega 6 fatty acids. In preferred embodiments calcium is present in a weight percent of about 3-50%. In other preferred embodiments, vitamin B-12 is present in a weight percent of about 1-5%. In especially preferred embodiments, the zeolite is a clinoptilolite zeolite and in other embodiments it is a heulandite zeolite or a mordenite zeolite or mixtures of the same. In yet other embodiments, the zeolite is synthetic. Additionally, in other especially preferred embodiments, the zeolite is present in the composition as a powder. Preferably, the average particle size of the zeolite present is about 1 to 2000 microns, more preferably 1 to 1000 microns, and especially preferably about 500 microns.

In a second aspect, the invention provides methods for preventing or treating a disease comprising the step of administering a therapeutically effective amount of a composition comprising a zeolite as described herein. The methods extend to treating, for instance, the hangover effect of alcohol, diarrhea, heartburn, gastrointestinal disease, toxic poisoning, influenza, and the common cold.

In a third aspect, the invention provides methods for detoxifying an organism or a biological sample. The target toxins to be removed may include for instance, natural toxins produced by bacteria such as those involved in pathogenesis of bacterial diseases, radiation particles, chemical toxins such as may be ingested, metabolic byproducts such as harmful substances involved in alcohol metabolism, as well as elements such as lead, arsenic, zinc and cesium. It is contemplated that the compositions of the invention may be used therapeutically by administering them to a living organism. Likewise, they may be used outside a living organism to detoxify a biological fluid, such as, for instance in vitro procedures or in conjunction with one or more apparatus for filtering a biological fluid such as blood.

In yet a fourth aspect, the invention provides methods for increasing vitamin or mineral absorption in vivo in an organism comprising the step of administering a therapeutically effective amount of the compositions described herein.

In yet a fifth aspect, the present invention features a kit comprising a zeolite containing composition as described herein. The kit may be intended for use in the treatment of one or more of hangover effects of alcohol, diarrhea, heartburn, gastrointestinal disease, toxic poisoning, influenza, and the common cold. Optionally, the kit may contain instructions for dosage and intervals between ingestion to maintain a therapeutically effective in vivo concentration of the active ingredients. The zeolite composition may be packaged in, for instance, a tablet, capsule, powder or solution form.

In a sixth aspect, the present invention features a nutritional supplement comprising a zeolite containing composition as described herein. The nutritional supplement may be used prophylactically or upon the advent of symptoms for the indications as described herein including treating hangover, diarrhea, influenza and the common cold. Moreover, individuals regularly exposed to environmental toxins may use the nutritional supplement to reduce the accumulation of such toxins in the body.

### DETAILED DESCRIPTION

Before the present methods and treatment methodology are described, it is to be understood that this invention is not limited to particular methods, and experimental conditions described, as such methods and conditions may vary. It is also to be understood that the terminology used herein is for purposes of describing particular embodiments only, and is not intended to be limiting, since the scope of the present invention will be limited only in the appended claims.

As used in this specification and the appended claims, the singular forms “a”, “an”, and “the” include plural references unless the context clearly dictates otherwise. Thus, for example, references to “the method” includes one or more methods, and/or steps of the type described herein and/or which will become apparent to those persons skilled in the art upon reading this disclosure and so forth.

Unless defined otherwise, all technical and scientific terms used herein have the same meaning as commonly understood by one of ordinary skill in the art to which this invention belongs. Although any methods and materials similar or equivalent to those described herein can be used in the practice or testing of the invention, the particular methods and materials are now described. All publications mentioned herein are incorporated herein by reference.

Definitions

The terms used herein have the meanings recognized and known to those of skill in the art; however, for convenience and completeness, particular terms and their meanings are set forth below.

“Treatment” refers to the administration of a drug or the performance of procedures with respect to a subject, for either prophylaxis (prevention) or to cure the infirmity or malady in the instance where the subject is afflicted.

By “individual” or “patient” or “subject” is meant a human or non-human mammal that may benefit from the therapeutic compositions and the therapies described in the present application, for example, a hangover due to alcohol ingestion, influenza infection, a common cold, or diarrhea.

By “average particle size” is meant that about 50% of the number of particles have a particle size less than or equal to the value mentioned and about 50% of the number of particles have a size greater then or equal to the value mentioned.

By “detoxifying” is meant removing a chemical compound or element from a biological sample or an organism or effectively neutralizing all or a part of the negative effects of the compound or element.

Description of the Therapeutic and Prophylactic Compositions and Their Use

The compositions of the present invention have therapeutic utility. It has been found that a composition comprising a zeolite is useful for treating multiple conditions. The zeolite is normally present in a weight percent of about 50-95%, preferably about 70-95%, and more preferably about 90-95%. Optionally, one or more other ingredients are present in the composition. Such supplemental ingredients may include one or more of calcium, vitamin B-12, vitamin A, vitamin B1, vitamin B2, vitamin B3, vitamin B6, vitamin B7, folate, pantothenic acid, vitamin C, vitamin D, vitamin E, vitamin K, iron, zinc, magnesium, patassium, boron, iodine, manganese, selenium, copper, chromium, omega 3 or omega 6 fatty acids. Calcium is normally present in a weight percent of about 3-50%. Vitamin B-12 may be present in a weight percent of about 1-5%. The zeolite is normally a clinoptilolite zeolite or a heulandite zeolite, however, it may be a synthetic form of a zeolite, and it is usually present in the composition as a powder having an average particle size of about 1 to 2000 microns, often about 500 microns.

The compositions described herein are useful for preventing or treating a disease such as the hangover effect of alcohol, diarrhea, heartburn, gastrointestinal disease, toxic poisoning, influenza, and the common cold. The compositions are further useful for detoxifying an organism or a biological sample. In addition, the compositions may be used as a dietary supplement.

The zeolite is substantially purified (e.g., substantially free from substances that limit its effect or produce undesired side-effects). Such zeolite may be obtained, for instance, as a clinoptilolite from Agricola Metals, Princeton, N.J. Zeolite is normally commercially available in about 88-92% pure form. The subject is preferably an animal, including but not limited to animals such as monkeys, cows, pigs, horses, chickens, cats, dogs, etc., and is preferably a mammal, and most preferably human. In one specific embodiment, a non-human mammal is the subject. In another specific embodiment, a human mammal is the subject.

Various delivery systems are known and can be used to administer the compositions of the invention, e.g., enteral or parenteral routes. Most commonly, oral routes are used such as a tablet or capsule or even as a powder that may be mixed with food or with a liquid.

Such compositions comprise a therapeutically effective amount of a zeolite and one or more optional ingredients, and optionally, a pharmaceutically acceptable carrier. In a particular embodiment, the term “pharmaceutically acceptable” means approved by a regulatory agency of the Federal or a state government or listed in the U.S. Pharmacopeia or other generally recognized pharmacopeia for use in animals, and more particularly in humans. The term “carrier” refers to a diluent, adjuvant, excipient, or vehicle with which the therapeutic is administered. Such pharmaceutical carriers can be sterile liquids, such as water and oils, including those of petroleum, animal, vegetable or synthetic origin, such as peanut oil, soybean oil, mineral oil, sesame oil and the like. Water is a preferred carrier when the pharmaceutical composition is administered intravenously. Saline solutions and aqueous dextrose and glycerol solutions can also be employed as liquid carriers, particularly for injectable solutions. Suitable pharmaceutical excipients include starch, glucose, lactose, sucrose, gelatin, malt, rice, flour, chalk, silica gel, sodium stearate, glycerol monostearate, talc, sodium chloride, dried skim milk, glycerol, propylene, glycol, water, ethanol and the like. The composition, if desired, can also contain minor amounts of wetting or emulsifying agents, or pH buffering agents. These compositions can take the form of solutions, suspensions, emulsion, tablets, pills, capsules, powders, sustained-release formulations and the like. The composition can be formulated as a suppository, with traditional binders and carriers such as triglycerides. Oral formulation can include standard carriers such as pharmaceutical grades of mannitol, lactose, starch, magnesium stearate, sodium saccharine, cellulose, magnesium carbonate, etc. Examples of suitable pharmaceutical carriers are described in “Remington's Pharmaceutical Sciences” by E. W. Martin. Such compositions will contain a therapeutically effective amount of the zeolite and one or more optional ingredients, preferably in purified form, together with a suitable amount of carrier so as to provide the form for proper administration to the subject. The formulation should suit the mode of administration.

Generally, the ingredients are supplied either separately or mixed together in unit dosage form, for example, as a dry lyophilized powder or water free concentrate in a hermetically sealed container such as an ampoule or sachette indicating the quantity of active agent. Where the composition is to be administered by infusion, it can be dispensed with an infusion bottle containing sterile pharmaceutical grade water or saline. Where the composition is administered by injection, an ampoule of sterile water for injection or saline can be provided so that the ingredients may be mixed prior to administration.

The amount of the zeolite and one or more optional ingredients of the invention effective in the treatment of the indicated disease or pathology can be determined by standard clinical techniques based on the present description. In addition, in vitro assays may optionally be employed to help identify optimal dosage ranges. The precise dose to be employed in the formulation will also depend on the route of administration, and the seriousness of the disease or disorder, and should be decided according to the judgment of the practitioner and each subject's circumstances. Effective doses may be extrapolated from dose-response curves derived from in vitro or animal model test systems. One of ordinary skill in the art will appreciate that the dosages and concentrations may be varied and the effect of the variations guaged with only routine experimentation. The dosage or concentration of the zeolite and other ingredients may be varied in accordance with a patient's disease type, severity, age, body build and response to treatment.

The invention also provides a pharmaceutical pack or kit comprising one or more containers filled with one or more of the ingredients of the pharmaceutical compositions of the invention. The ingredients may be in the form of capsules or tablets, for instance. Optionally associated with such container(s) can be a notice in the form prescribed by a governmental agency regulating the manufacture, use or sale of pharmaceuticals or biological products, which notice reflects (a) approval by the agency of manufacture, use or sale for human administration, (b) directions for use, or both.

If delivery is contemplated in tablet or capsule form, natural minerals containing a zeolite such as a clinoptilolite or heulandite or mixtures thereof are substantially purified by washing. The zeolites are crushed to a particle size of not greater than 0.1 mm and preferably about 500 microns in size. A permanent magnet may be used to remove any foreign inclusions from the zeolites. Another concern is that due to the negative charge of minerals present in the zeolite. Heavy metals that have a positive charge and come in contact with the zeolite are attracted to the molecule. In order to release the heavy metals, the clinoptilolite may be washed in a solution such as a salt water solution. Sodium ions therein replace the heavy metals attracted to the clinoptilolite. After washing with the high sodium solution, the zeolite may then be washed with clean water to remove excess sodium. Afterwards, the zeolites may be put in temperature controlled drying cabinets to be dehydrated. Zeolites may be calcined at, for instance, 200 to 300° C., or even up to 650° C. After calcination, the zeolite may be mixed with other components that are commercially available in metered amounts specified for the formulation and mixed to form a homogeonous formulation. Prior to packaging, further sterilization procedures may be performed such as, for example heating to approximately 120° C. The composition may be encapsulated in capsule form according to standard procedures known in the art such as those used by, for example, Bactolac Pharmaceuticals.

### DETAILED DESCRIPTION OF THE PREFERRED EMBODIMENTS

### EXAMPLE 1

A composition in capsule form was prepared containing 800 mg of clinoptilolite zeolite, 50 mcg vitamin-B12 (cyanocobalamin) and 72 mg calcium (calcium carbonate) according to standard encapsulation techniques. Individuals suffering from the effects of or anticipating that they may suffer from the effects of hangover take approximately six capsules either before or during the period in which they are ingesting alcoholic beverages.

### EXAMPLE 2

A composition in capsule form was prepared containing 800 mg of clinoptilolite zeolite, 50 mcg vitamin-B12 (cyanocobalamin) and 72 mg calcium (calcium carbonate) according to standard encapsulation techniques. Individuals suffering from diarrhea, either acute or chronic, take approximately three to six capsules either during the period in which they are experiencing diarrhea or on a regular basis to prevent or ameliorate diarrhea.

### EXAMPLE 3

A composition in capsule form was prepared containing 800 mg of clinoptilolite zeolite, 50 mcg vitamin-B12 (cyanocobalamin) and 72 mg calcium (calcium carbonate) according to standard encapsulation techniques. Individuals suffering from symptoms of influenza or common cold take approximately six capsules approximately three times daily during the period in which they are symptomatic.

1. A pharmaceutical composition of therapeutic utility comprising a zeolite in a weight percent of about 50-95%.

2. A pharmaceutical composition according to claim 1 further comprising vitamin B-12.

3. A pharmaceutical composition according to claim 1 further comprising calcium.

4. A pharmaceutical composition according to claim 1 further comprising one or more ingredients selected from the group consisting of calcium, vitamin B-12, vitamin A, vitamin B1, vitamin B2, vitamin B3, vitamin B6, vitamin B7, folate, pantothenic acid, vitamin C, vitamin D, vitamin E, vitamin K, iron, zinc, magnesium, potassium, boron, iodine, manganese, selenium, copper, chromium, and omega 3 or omega 6 fatty acids.

5. The pharmaceutical composition of claim 3 wherein the calcium is present in a weight percent of about 3-50%.

6. The pharmaceutical composition of claim 2 wherein the vitamin B-12 is present in a weight percent of about 1-5%.

7. The pharmaceutical composition of claim 1 wherein the zeolite is a clinoptilolite zeolite.

8. The pharmaceutical composition of claim 1 wherein the zeolite is a heulandite zeolite.

9. The pharmaceutical composition of claim 1 wherein the zeolite is present in the composition as a powder having an average particle size of 1 to 1000 microns.

10. A method for preventing or treating a disease comprising the step of administering a therapeutically effective amount of a composition comprising a zeolite in a weight percent of about 50-90%.

11. The method according to claim 10 wherein the disease is the hangover effect of alcohol.

12. The method according to claim 10 wherein the disease is diarrhea.

13. The method according to claim 10 wherein the disease is heartburn.

14. The method according to claim 10 wherein the disease is a gastrointestinal disease.

15. The method according to claim 10 wherein the disease is toxic poisoning.

16. The method according to claim 10 wherein the disease is influenza.

17. The method according to claim 10 wherein the disease is the common cold.

18. A method for detoxifying an organism or a biological sample comprising the step of administering a composition comprising a zeolite in an amount of about 50-95 weight percent, calcium in an amount of about 3-50 weight percent and one or more ingredients selected from the group consisting of vitamin B-12, vitamin A, vitamin B1, vitamin B2, vitamin B3, vitamin B6, vitamin B7, folate, pantothenic acid, vitamin C, vitamin D, vitamin E, vitamin K, iron, zinc, magnesium, potassium, boron, iodine, manganese, selenium, copper, chromium, and omega 3 or omega 6 fatty acids.

19. The method according to claim 18 wherein the detoxifying is performed to remove one or more toxins selected from the group consisting of natural toxins produced by bacteria, an ingested chemical toxin, and metabolic byproducts.

20. A method for increasing vitamin or mineral absorption in vivo in an organism comprising the step of administering a therapeutically effective amount of a composition comprising a zeolite in an amount of about 50-95 weight percent, calcium in an amount of about 3-50 weight percent and one or more ingredients selected from the group consisting of vitamin B-12, vitamin A, vitamin B1, vitamin B2, vitamin B3, vitamin B6, vitamin B7, folate, pantothenic acid, vitamin C, vitamin D, vitamin E, vitamin K, iron, zinc, magnesium, potassium, boron, iodine, manganese, selenium, copper, chromium, omega 3, and omega 6 fatty acids.

21. A nutritional supplement of therapeutic utility comprising a zeolite in a weight percent of about 50-95%.

22. A nutritional supplement according to claim 21 further comprising vitamin B-12.

23. A nutritional supplement according to claim 21 further comprising calcium.

24. A nutritional supplement according to claim 21 further comprising one or more ingredients selected from the group consisting of calcium, vitamin B-12, vitamin A, vitamin B1, vitamin B2, vitamin B3, vitamin B6, vitamin B7, folate, pantothenic acid, vitamin C, vitamin D, vitamin E, vitamin K, iron, zinc, magnesium, potassium, boron, iodine, manganese, selenium, copper, chromium, and omega 3 or omega 6 fatty acids.

25. The nutritional supplement of claim 23 wherein the calcium is present in a weight percent of about 3-50%.

26. The nutritional supplement of claim 22 wherein the vitamin B-12 is present in a weight percent of about 1-5%.

27. The nutritional supplement of claim 21 wherein the zeolite is a clinoptilolite zeolite.

28. The nutritional supplement of claim 21 wherein the zeolite is a heulandite zeolite.

29. The nutritional supplement of claim 21 wherein the zeolite is present in the composition as a powder having an average particle size of 1 to 1000 microns.

30. A kit having a composition comprising a zeolite in an amount of about 50-95 weight percent, calcium in an amount of about 3-50 weight percent and one or more ingredients selected from the group consisting of vitamin B-12, vitamin A, vitamin B1, vitamin B2, vitamin B3, vitamin B6, vitamin B7, folate, pantothenic acid, vitamin C, vitamin D, vitamin E, vitamin K, iron, zinc, magnesium, potassium, boron, iodine, manganese, selenium, copper, chromium, and omega 3 or omega 6 fatty acids.

31. The kit according to claim 30 intended for use in treating one or more of hangover effects of alcohol, diarrhea, heartburn, gastrointestinal disease, toxic poisoning, influenza, and the common cold.

32. The kit according to claim 30 wherein the composition is in a capsule.

33. The kit according to claim 30 wherein the composition is in a tablet.
